(19) United States
(12) Patent Application Publication
(10) Pub. No.: US 2017/0315141 A1
(43) Pub. Date: Nov. 2, 2017

(54) METHODS FOR BLOOD TYPING AND ANTIBODY SCREENING

(71) Applicant: T2 Biosystems, Inc., Lexington, MA (US)

(72) Inventors: Thomas Jay LOWERY, JR., Belmont, MA (US); Vyacheslav PAPKOV, Waltham, MA (US); Walter W. MASSEFSKI, JR., Sharon, MA (US)

(21) Appl. No.: 15/526,086

(22) PCT Filed: Nov. 12, 2015
(86) PCT No.: PCT/US15/60340

§ 371 Date: May 11, 2017

Related U.S. Application Data
(60) Provisional application No. 62/079,419, filed on Nov. 13, 2014.

(51) Int. Cl. G01N 33/80; G01N 33/542

(52) U.S. Cl. CPC G01N 33/80; G01N 33/542

(57) ABSTRACT

The invention features systems and methods for the detection of analytes, particularly antigens, complement, and antibodies present in blood. The invention features a system for the detection of antigens, complement, and antibodies on the surface of RBCs and circulating antibodies in the blood.

### BACKGROUND OF THE INVENTION

This invention features assays for the detection of analytes in blood and their use in medical treatment and diagnosis of disease.

Blood is the circulating tissue of an organism that carries oxygen and nutritive materials to the tissues and removes carbon dioxide and various metabolic products for excretion. Whole blood consists of a pale yellow or gray yellow fluid, plasma, in which are suspended red blood cells (RBCs), white blood cells, and platelets. Red blood cells have proteins and sugars on their surfaces that have antigenic properties that can stimulate the production of antibodies.

Blood testing for various antigens and antibodies is commonly performed in clinical laboratories. Specifically, the blood must be typed to determine the identity of antigens on the surface of the RBCs, and also determine whether there are any antibodies to erythrocyte antigens present in the serum or on the surface of RBCs. Traditionally blood typing, compatibility testing, and antibody screening have been done through some type of agglutination assay involving antigen-antibody reactions.

Currently, 35 different blood group systems are known, nine of which are considered the major blood group systems. Additionally, there are various blood group antigens of low or high frequency. There are over 600 known blood group antigens with over 1000 different associated genetic alleles. These antigens are generally either sugars or proteins. The prominent ABO system was discovered in 1900 by Landsteiner when he observed that serum from healthy humans could cause agglutination of the red blood cells of other individuals. The other nine important blood groups are MNS, P, Rh, Lutheran, Kell, Lewis, Duffy and Kidd. The A and B antigens of the ABO system and the D antigen of the Rh system have greater immunogenicity than virtually all other red cell antigens. They are therefore most frequently screened, as severe complications can result from improper transfusions involving RBCs with these antigens.

The ABO erythrocyte antigens are dependent on the presence of both the H gene and the ABO genes. These genes code for glycosyltransferases. The H-transferase adds L-fucose to a precursor carbohydrate chain present on the erythrocyte membrane. After the L-fucose transfer, the A- and B-transferases can act if present, adding N-acetyl-D-galactosamine, or D-galactose respectively. Healthy adults who lack a particular ABO group antigen on their RBCs usually have the corresponding antibody in their serum as a result of stimulation from the environment, such as contact with bacteria or food that contain A-, B-, or H-like substances. Thus, these antibodies can cause a potent immune response if an individual has contact with these antigens through pregnancy, an accidental transfusion, autoimmune diseases, or by some other means.

Red blood cells are flexible, oval bioconcave disks that contain hemoglobin, an iron-containing protein that binds oxygen. The RBC membrane contains proteins and glycoproteins embedded in a fluid lipid bilayer. Sialyted glycoproteins of the membrane are responsible for causing the surface of the RBC to be negatively charged with a zeta potential that induces repulsion between cells. The charges help prevent the interaction between individual RBCs and other cells. Mechanisms that reduce the zeta potential of RBCs can promote hemagglutination reactions.

Red blood cells can form stacks called rouleaux, which form due to the unique discoid shape of the cells. These formations occur when the plasma protein concentration is high, and they cause an increase in the erythrocyte sedimentation rate. Infections, multiple myeloma, inflammatory and connective tissue disorders, and cancers can lead to rouleaux formation, and as such it is a non-specific indicator of disease. Acute phase proteins such as fibrinogen interact with sialic acid on the surface of RBCs to induce rouleaux. It can also be caused by an increase in the ratio of erythrocytes to plasma volume, and it is slowed by the presence of albumin proteins. Rouleaux formation is the most common cause of false positive tests for hemagglutination.

There are five classes of immunoglobulins or antibodies. They are IgM, IgG, IgA, IgD, and IgE. All of these can be present in blood, but IgM and IgG are the most relevant to blood typing and hemagglutination reactions, though IgA antibodies to A, B, and D antigens can exist.

IgM is usually the first immunoglobulin produced as an initial response to an insult. It is a large molecule of approximately 900 KD in mass that is mainly confined to the bloodstream. It normally consists of five monomers joined in a cyclic pentameric structure that has ten possible but five readily available sites to bind with an antigen. When IgM red blood cell antibodies are mixed with a suspension of erythrocytes bearing the corresponding antigens, each IgM molecule is large enough to simultaneously bind to red cell antigen sites on adjacent red cells resulting in hemagglutination.

Approximately 80% of circulating antibodies are IgG, a smaller immunoglobulin (approximately 160 KD) that can enter tissues. Each IgG molecule has two antigen-binding sites, but in practice the molecule usually acts as a monomer that coats or sensitizes a single antigen on a single cell. Most classes of IgG can cross the placenta and can activate complement.

The complement system is part of the innate immune system. It consists of a number of small proteins that normally circulate as inactive precursors. When triggered, proteolytic enzymes cleave specific proteins to release cytokines and initiate a massive cascade and activation of the cell-killing membrane attack complex. Complement can be activated by antigen-antibody reactions and by bacterial polysaccharides, viruses, enzymes, and endotoxins.

There are serious medical conditions that lead to antibodies, complement, or both antibodies and complement system factors being bound to the erythrocyte or RBC surface antigens in vivo. These bound antibodies are associated with various diseases in which an immune mechanism is attacking the patient's own erythrocytes. These mechanisms can be caused by autoimmunity, alloimmunity, or drug-induced immune-mediated reaction. These conditions can include hemolytic disease of the newborn, autoimmune hemolytic anemia, lupus erythematosus, hemolytic transfusion reactions, and drug-induced autoimmune hemolytic anemia.

The identification of antibodies in serum frequently involves the use of expensive donor blood cell reagents, and the use of less of these reagents in a high throughput fashion would be advantageous.

### SUMMARY OF THE INVENTION

The invention features systems and methods for the detection of analytes, particularly antigens, complement, and antibodies present in blood. The invention features a system for the detection of antigens, complement, and antibodies on the surface of RBCs and circulating antibodies in the blood.

In an first aspect, the invention features a method for identifying the presence of an analyte in a sample, the method including: (a) providing red blood cells substantially free of antibodies (i.e., containing insufficient amounts of antibodies to interfere with the method) and contacting the red blood cells with a primary antibody capable of complexing to the red blood cells when the red blood cells include an antigen for the primary antibody; (b) following step (a), (i) combining the red blood cells with an aqueous solution to form an aqueous sample, wherein the aqueous sample is substantially free of uncomplexed antibodies, and (ii) contacting the aqueous sample with a multivalent binding agent, wherein the multivalent binding agent is operative to promote aggregation of the red blood cells when the primary antibody is complexed to the red blood cells; (c) following step (b), placing the aqueous sample in a device, the device including a support defining a well configured to hold the aqueous sample, and having an RF coil disposed about the well, the RF coil configured to detect a magnetic resonance signal produced by exposing the aqueous sample to a bias magnetic field created using one or more magnets and an RF pulse sequence, and exposing the aqueous sample to a bias magnetic field and an RF pulse sequence; (d) following step (c), measuring the magnetic resonance signal; and (e) on the basis of the signal measured in step (d), determining whether the analyte is present, wherein the analyte is the primary antibody or a red blood cell including an antigen for the primary antibody. In particular embodiments, the red blood cells are obtained from a subject and the primary antibody is a reagent primary antibody selective for an antigen found on red blood cells. In still other embodiments, the primary antibody is obtained from a subject, and the red blood cells are reagent red blood cells that solely bear antigens for the primary antibody. The primary antibody can be obtained from a subject, and the red blood cells can be reagent red blood cells bearing antigens for a mixture of primary antibodies; in part (e) if antibodies are determined to be present, the method can be repeated with red blood cells solely bearing antigens for the primary antibody. Optionally, prior to step (a) the subject primary antibodies can be separated from any other plasma components (e.g., separated by using dried red blood cells displaying the corresponding antigen on their surfaces; or by using magnetic particles coated with the corresponding blood group antigen; or by any other method described herein). In particular embodiments of the method, the primary antibody and the antigen for the primary antibody are characteristic of the blood type of a subject, and step (f) includes determining the blood type of the subject.

In a related aspect, the invention features a method for identifying the presence of an analyte in a sample, the method including: (a) providing red blood cells substantially free of antibodies; (b) following step (a), (i) combining the red blood cells with an aqueous solution to form an aqueous sample, wherein the aqueous sample is substantially free of uncomplexed antibodies, and (ii) contacting the aqueous sample with a multivalent binding agent, wherein the multivalent binding agent is operative to promote aggregation of the red blood cells when it is complexed to the red blood cells; (c) following step (b), placing the aqueous sample in a device, the device including a support defining a well configured to hold the aqueous sample, and having an RF coil disposed about the well, the RF coil configured to detect a magnetic resonance signal produced by exposing the aqueous sample to a bias magnetic field created using one or more magnets and an RF pulse sequence, and exposing the aqueous sample to a bias magnetic field and an RF pulse sequence; (d) following step (c), measuring the magnetic resonance signal; and (e) on the basis of the signal measured in of step (d), determining whether the analyte is present, wherein the analyte is a red blood cell including an antigen able to bind to the multivalent binding agent. In particular embodiments of the method, the antigen on the surface of the red blood cells and the multivalent binding agent selective for that antigen are characteristic of the blood type of a subject, and step (f) includes determining the blood type of the subject.

In another aspect, the invention features a method for testing the blood of a subject, the method including: (a) providing a sample including red blood cells from the subject, the red blood cells potentially being bound with at least one of antibodies and complement factors; (b) following step (a), (i) combining the red blood cells with an aqueous solution to form an aqueous sample, wherein the aqueous sample is substantially free of uncomplexed antibodies and complement factors, and (ii) contacting the aqueous sample with a multivalent binding agent, wherein the multivalent binding agent is operative to promote aggregation of the red blood cells when at least one of the primary antibody and complement factors is complexed to the red blood cells; (c) following step (b), placing the aqueous sample in a device, the device including a support defining a well configured to hold the aqueous sample including the red blood cells and antibodies, and having an RF coil disposed about the well, the RF coil configured to detect a magnetic resonance signal produced by exposing the aqueous sample to a bias magnetic field created using one or more magnets and an RF pulse sequence, and exposing the aqueous sample to a bias magnetic field and an RF pulse sequence; (d) following step (c), measuring the magnetic resonance signal; and (e) on the basis of the signal measured in of step (d), determining the blood disease. In one embodiment of the method, the red blood cells are separated by washing (e.g., by centrifugation). In another embodiment of the method, no multivalent binding agent is used, but red blood cells are mixed to promote rouleaux and aggregation.

In one embodiment of any of the above methods, prior to step (b)(i) the red blood cells are separated from any uncomplexed antibodies. The red blood cells can be separated by the use of sepharose or gel size-exclusion beads; by utilizing fibrin mesh (e.g., a fibrin mesh is formed by adding reptilase); by use of a synthetic gel; can be separated via a fluidic system; separated by utilizing magnetic particles labeled with multivalent binding agents that complex red blood cells via alternate antigens from the primary antibody; or separated by utilizing a microplate based technique.

In another embodiment of any of the above methods, excess multivalent binding agent is added in step (b)(ii) to overcome the effects of any uncomplexed primary antibody.

In a related aspect, the invention features a method for identifying the presence of a primary antibody in a subject serum or plasma, the method including: (a) providing an aqueous sample including reagent red blood cells complexed with a reagent primary antibody and a selective multivalent binding agent, wherein the red blood cells form an aggregate with the primary antibody and the selective multivalent binding agent; (b) contacting the aqueous sample with solution including a subject serum or plasma, wherein the subject serum or plasma contains a primary antibody that is operative to promote disaggregation of the reagent red blood cells when the subject antibody complexes the selective multivalent binding agent; (c) placing the aqueous sample in a device, the device including a support defining a well configured to hold the aqueous sample, and having an RF coil disposed about the well, the RF coil configured to detect a magnetic resonance signal produced by exposing the aqueous sample to a bias magnetic field created using one or more magnets and an RF pulse sequence and exposing the aqueous sample to a bias magnetic field and an RF pulse sequence; (d) following step (c), measuring the magnetic resonance signal; and (e) on the basis of the signal measured in of step (d), determining whether the antibody is present. In particular embodiments of the method, the primary antibody and the antigen for the primary antibody are characteristic of the blood type of a subject, and step (f) includes determining the blood type of the subject.

In one embodiment of any of the above methods, the aqueous sample includes from 0.5-20% hematocrit (e.g., from 0.5-2%, 2-4%, 4-8%, 8-12%, or 12-20% hematocrit).

In another embodiment of any of the above methods, the aqueous sample includes from 1 to 50 microliters (e.g., from 1-5, 5-15, 15-25, or 5-40, or 25-50 microliters).

In particular embodiments of the above methods, the primary antibody and the antigen for the primary antibody are characteristic of the blood type of a subject, and step (f) includes determining the blood type of the subject.

In one embodiment of any of the above methods, the method further includes adding in step (b)(i), a potentiator. The potentiator can be any potentiator described herein.

In another embodiment of any of the above methods, the multivalent binding agent is monoclonal IgM, antihuman globulin reagent, or is selective for the primary antibody. The multivalent binding agent can be an aptamer; an RNA, DNA, or XNA aptamer; a polypeptide aptamer; peptidomimetic aptamer; a secondary antibody; a dendrimer; or a Fab fragment.

In particular embodiments of the above methods, the reagent red blood cells are single donor group ◯ red blood cells.

In another embodiment of any of the above methods, the multivalent binding agent includes a magnetic particle.

In one embodiment of any of the above methods, in step (b)(ii) the multivalent binding moiety is titrated into the red blood cells.

In particular embodiments of the above methods, there is dynamic mixing during step (c).

In another embodiment of any of the above methods, fibrin mesh is used during step (c) to suspend the red blood cells and prevent erythrocyte sedimentation.

In one embodiment of any of the above methods, a synthetic gel is used during step (c) to suspend the red blood cells and prevent erythrocyte sedimentation.

In another embodiment of any of the above methods, an additive is added in step (c) to increase viscosity and prevent erythrocyte sedimentation.

In particular embodiments of the above methods, the aqueous sample is centrifuged prior to step (c), in order to promote aggregation.

In one embodiment of any of the above methods, the uncomplexed red blood cells are recovered for reuse after the assay is completed.

In particular embodiments of the above methods, proteolytic enzymes are added prior to step (b)(ii).

In particular embodiments of the above methods, the red blood cells can be complexed with or sensitized by complement and the multivalent binding agent is operative to bind to complement and induce aggregation of the red blood cells. The multivalent binding agent can be antihuman globulin reagent.

In another embodiment of any of the above methods, the method further includes (i) making a series of magnetic resonance relaxation rate measurements of water in the aqueous sample; (ii) transforming the measurements using an algorithm that distinguishes two or more separate water populations within the aqueous sample, wherein each separate water population is characterized by one or more magnetic resonance parameters having one or more values; and (iii) on the basis of the transformed measurements of step (ii), determining the presence and identity of antigens on the surface of red blood cells in the blood sample and additionally or alternatively determining the presence of antibodies in the aqueous sample.

As used herein, the term “primary antibody” refers to an immunoglobulin molecule with specificity for a particular epitope and it binds that epitope directly.

As used herein, the term “specific” or “specificity” when referring to an interaction between an antigen and an antibody or multivalent binding agent means, for example, that the antibody or multivalent binding agent selectively binds the particular antigen (e.g. blood group antigen) with an interaction that is measurably stronger than a non-specific interaction. For example, this interaction can be measured through the use of in vitro assays with a target and exhibit a binding constant of less than or equal to K_{d}=1×10⁻⁴ (e.g. 10⁻⁴, 10⁻⁵, 10⁻⁶, 10⁻⁷, 10⁻⁸, 10⁻⁹, 10⁻¹⁰, 10⁻¹¹, 10⁻¹², or less). In one example, the antibody or multivalent binding agent selectively binds the particular blood group antigen with greater affinity (e.g. 2-fold, 5-fold, 10-fold, 100-fold, or greater) than it binds the alternate blood group antigens. In another example, the antibody specific for a particular blood group antigen is one that will cause aggregation of RBCs bearing that antigen on their surfaces in the possible presence of RBCs bearing an alternate antigen on their surfaces.

As used herein, the term “secondary antibody” is an immunoglobulin molecule that binds one or more primary antibodies.

As used herein, the term “reagent primary antibody” refers to a primary antibody that is not part of the subject sample but is provided as a reagent. For example, the reagent primary antibody is specific for a certain blood group antigen.

As used herein, the term “multivalent binding agent” refers to an agent that can simultaneously bind more than one molecule. Multivalent binding agents include, for example, antibodies and aptamers.

As used herein, the term “reagent red blood cells” are red blood cells that have been characterized for their antigen profiles. Reagent red blood cells can be obtained from a single donor or a mixture of donors.

As used herein, the term “aggregation” refers to the clumping of particles, cells, or cells and particles. Aggregation is mediated by the interactions of antibodies or other multivalent binding agents.

As used herein, the term “potentiator” refers to a reagent that enhances sensitization of an antigen and the aggregation of red blood cells. Examples of potentiators include, without limitation, albumin, low ionic-strength saline, and polyethylene glycol.

As used herein, the term “aptamer” refers to an oligomer that binds to a specific target molecule. Aptamers include XNA aptamers and peptidomimetic aptamers.

As used herein, the term “XNA aptamer” refers to a synthetic polymer including nucleic acid analogs that binds to a specific epitope. XNA aptamers can formed from, for example, peptide nucleic acids, locked nucleic acids, glycol nucleic acids, threose nucleic acids, and/or nucleic acids with a phosporothioate backbone.

As used herein, the term “peptidomimetic aptamer” refers to a small protein-like chain designed to mimic a peptide that binds to a specific epitope. Peptidomimetic aptamers can be formed, for example, by modification of an existing peptide, or by de novo design and can include peptoids and β-peptides.

As used herein, the term “dendrimer” refers to a repetitively branched molecule.

As used herein, the term “fab fragment” refers to a region on an antibody that binds to antigens.

As used herein, the term “fibrin mesh” refers to polymerized fibrin.

As used herein, “reptilase” refers to a serine protease from Bothrops atrox that is a thrombin-like proteolytic enzyme that releases fibrinopeptide A from fibrinogen and leads to clot formation. Reptilase is also known as batroxobin.

As used herein, the term “anti-human globulin reagent” refers to anti-human antibodies otherwise known as Coombs reagent. Anti-human globulin can be a mixture of different anti-human antibodies, or can be monospecific for IgG, IgA, IgM, C3, C3d, C4, or other human antibody or complement component.

As used herein, the term “NMR relaxation rate” refers to any of the following in a sample: T₁, T₂, T₁ᵣₕₒ, T₂ᵣₕₒ, and T₂*. NMR relaxation rates may be measured and/or represented using T₁/T₂ hybrid detection methods. Additionally, apparent diffusion coefficient (ADC) can be determined and evaluated (Vidmar et al. NMR in BioMedicine, 2009; and Vidmar et al., Eur. Biophys. J. 2008).

As used herein, the term “T₁/T₂ hybrid” refers to any detection method that combines a T₁ and a T₂ measurement. For example, the value of a T₁/T₂ hybrid can be a composite signal obtained through the combination of, ratio, or difference between two or more different T₁ and T₂ measurements. The T₁/T₂ hybrid can be obtained, for example, by using a pulse sequence in which T₁ and T₂ are alternatively measured or acquired in an interleaved fashion. Additionally, the T₁/T₂ hybrid signal can be acquired with a pulse sequence that measures a relaxation rate that is comprised of both T₁ and T₂ relaxation rates or mechanisms.

As used herein, the term “T₂ signature” refers to a curve established by applying a mathematical transform (e.g., a Laplace transform or Inverse Laplace Transform) to a decay curve associated with a relaxation rate parameter at a discrete time point or over a set time duration during a rheological event. T₂ signature curves provide information about the relative abundance of multiple water populations in a sample. As aggregation or disaggregation of RBCs, particles, or RBCs and particles progresses, the T₂ signature curves will reflect the changes within the sample. T₂ signatures may be used advantageously to assess, in real time, a discriminated hemostatic condition of a subject. Further, a T₂ signature may be a two dimensional (intensity versus T₂ value or T₂ value versus time) or three dimensional representation (intensity versus T₂ value versus time). The T₂ values in the two- or three dimensional representation may be replaced with or compared to other NMR signals such as T₁, T₁/T₂ hybrid, T₁ᵣₕₒ, T₂ᵣₕₒ and T₂*.

As used herein, the term “hematocrit” refers to the percentage, by volume, of red blood cells in a whole blood sample.

As used herein, the term “RBC ghost” refers to a dried red blood cell reagent that comprises mostly spectrin, a cytoskeletal protein including antigens on the surface of the RBC ghost.

Other features and advantages of the invention will be apparent from the following Detailed Description, the drawings, and the claims.

### BRIEF DESCRIPTION OF THE DRAWINGS

FIG. 1 is a scheme depicting an assay architecture of the invention. In this assay red blood cells are contacted by a primary antibody specific to an antigen on the surface of the red blood cells. A multivalent binding agent is added which binds to the primary antibody and induces aggregation of the red blood cells. The RBCs of FIG. 1 can be subject RBCs as part of a forward blood typing assay, or reagent RBCs as part of a reverse blood typing assay. The primary antibody can be a reagent primary antibody as part of a forward blood typing assay, or a subject primary antibody as part of a reverse blood typing assay.

FIG. 2 is a scheme depicting an assay architecture of the invention. In this assay, red blood cells are contacted by a multivalent binding agent that induces the aggregation of the red blood cells.

FIG. 3 is a scheme depicting an assay architecture of the invention. Red blood cells bound with antibodies are contacted with a multivalent binding agent that induces the aggregation of the red blood cells.

FIG. 4 is a scheme depicting an assay architecture of the invention. In this assay, red cells complexed with antibodies and specific multivalent binding moieties are contacted by primary antibody that induces the disaggregation of the red blood cells.

### DETAILED DESCRIPTION

The invention features systems and methods for the rapid detection of antibodies and antigens on the surface of red blood cells and antibodies to erythrocyte antigens present in serum or plasma. Determining the identity of these antibodies and antigens is essential for establishing the blood type of a patient and diagnosing a number of blood related disorders. The systems and methods of the invention can employ red blood cells, an NMR unit, optionally magnetic particles, optionally one or more magnetic assisted agglomeration units, optionally one or more incubation stations at different temperatures, optionally one or more vortexer, optionally one or more centrifuges, optionally a fluidic manipulation station, optionally a robotic system, and optionally one or more modular cartridges.

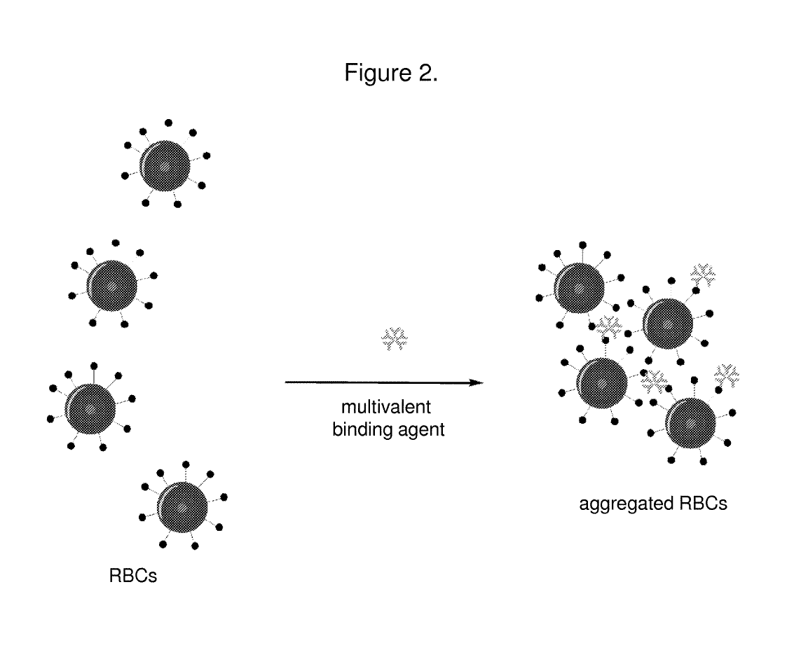

In one assay architecture of the invention with an embodiment depicted in FIG. 2, the blood type of subject erythrocytes are determined by mixing RBCs with a specific antibody that binds to cell-surface carbohydrate antigens. This leads to selective aggregation of the RBCs depending on their cell-surface antigens and the antibody type. This method relies on the detection of RBC antigens and is commonly referred to as forward blood typing.

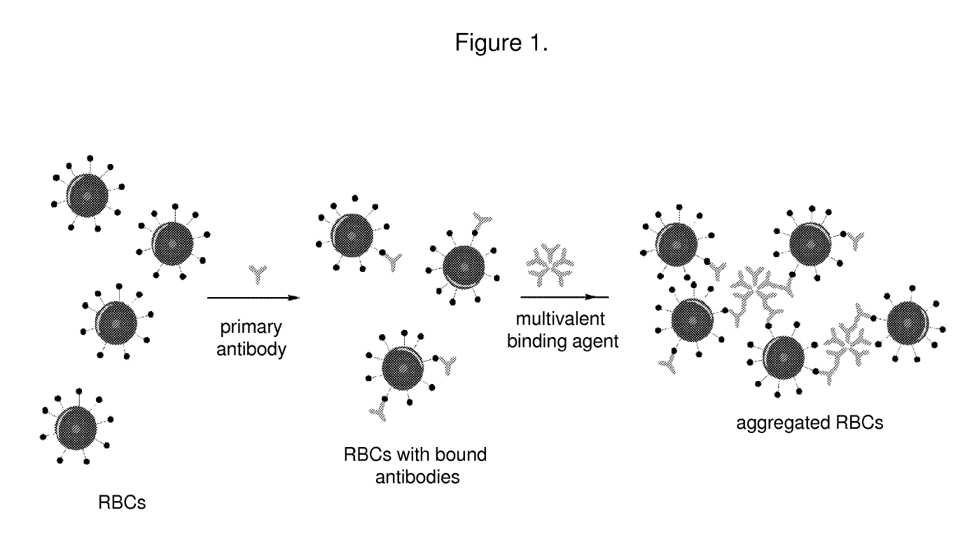

In another assay architecture of the invention with an embodiment depicted in FIG. 1, the assay screens for antibodies in the blood. Blood is screened for antibodies to any of over 600 RBC antigens. This approach requires the use of specialized mixtures of donor RBCs that present, as a mixture, or singly, all (or most) known blood types. This reagent composed of selected donor RBCs, known as reagent RBCs, is mixed with subject serum or plasma and processed to determine whether an antibody is present in the subject serum or plasma that will bind to the reagent RBCs.

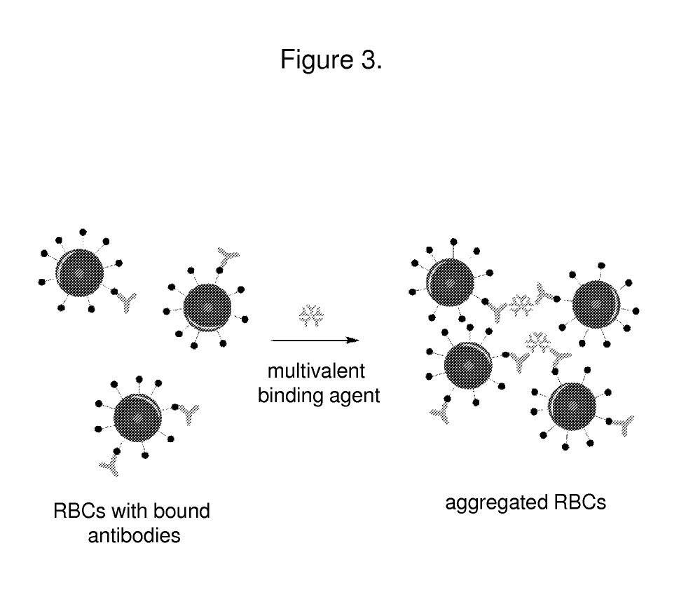

In still another assay architecture of the invention with an embodiment depicted in FIG. 3, certain blood disorders can be diagnosed by determining whether there are at least one of antibodies and complement on the surface of subject red blood cells. This is accomplished by mixing subject RBCs, potentially bound with at least one of antibodies and complement, with an antibody or multivalent binding agent that binds to antibodies and complement potentially on the surface of the RBCs and induces aggregation of the RBCs.

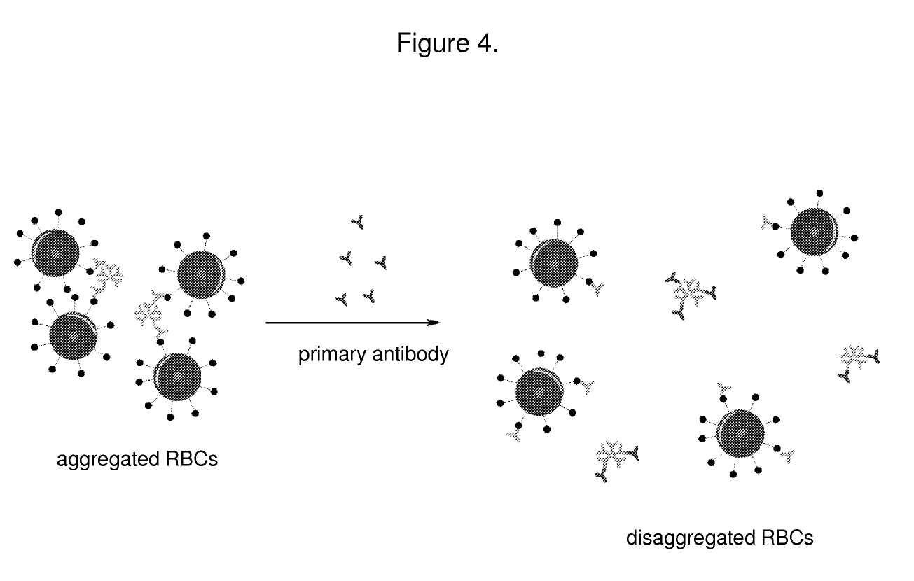

In yet another assay architecture of the invention with an embodiment depicted in FIG. 4, a disaggregation assay is used. Subject serum potentially containing antibodies is mixed with an aggregate of reagent RBCs bound with a specific multivalent binding agent. If disaggregation is observed, antibodies specific to the multivalent binding agent are present in the serum.

There are several different methods that can be employed to enhance aggregation of RBCs. Such methods reduce the negative charge of RBC surface molecules, reduce the hydration layer around the erythrocytes, or introduce positively charged macromolecules that aggregate the cells. The zeta potential of RBCs can be decreased in several ways. One way is by increasing the dielectric constant, or by changing the composition of the medium by adding macromolecular substances such as albumin, polyethylene glycol, polybrene, polyvinylpyrrolidone, gelatin, gum acacia, or dextran, among others. Low ionic strength saline solution (LISS) consisting of a salt solution with 20% less sodium chloride as compared to a normal isotonic solution can also be used. Putting an RBC in this solution reduces the ionic layer surrounding the erythrocyte and promotes the formation of antigen-antibody complexes.

Proteolytic enzymes can also be used to enhance aggregation reactions. Proteases such as bromelain (bromelin), papain, chymotrypsin, dispase, ficin, neuraminidase, pepsin, and trypsin are used. These proteases will remove glycoprotein fragments from the RBC membrane, allowing for greater proximity between RBCs and creating better access of antibodies to blood group antigens. The removal of sialic acid by some proteases directly reduces the electrical charge of the RBC surface reducing the zeta potential. Certain antigen-antibody reactions, such as Le^{a}, Le^{b}, I, P₁, and Rh, are enhanced by the addition of proteases. However, the addition of proteases is a secondary technique, as some antigens, such as M, N, S, Fy^{a}, Fy^{b}, and to some extent K, are not identifiable after protease treatment.

Multivalent binding agents can also promote aggregation of the red blood cells. Multivalent binding agents can include antibodies, antibody fragments, aptamers, dendrimers, and particles conjugated with any of the aforementioned molecules. Aptamers are oligomeric molecules engineered to bind to a molecular target of interest. Aptamers made of nucleic acids or nucleic acid analogs can be engineered through repeated rounds of in vitro selection. Peptidomimetic aptamers can be generated through combinatorial libraries constructed by phage display and other surface display technologies.

### Forward Blood Typing

A forward blood typing assay can ascertain the presence of A, B, O, and D antigens on the surface of RBCs. One embodiment of this concept is shown in FIG. 2. Erythrocytes from a subject sample are mixed with a multivalent binding agent that is specific for one of the blood types. After mixing, the RBCs will aggregate if the multivalent binding agent binds to the antigens on the surface of the RBCs. T₂ Magnetic Resonance (T2MR) can be utilized as an end-point reading after a fixed amount of time, or a kinetic assay can be performed as described below. Forward blood typing can also be performed by utilizing a primary antibody specific for the antigen on the surface of the RBCs. A multivalent binding agent capable of binding the primary antibody can then be added to induce aggregation of the RBCs. An example of this embodiment is shown in FIG. 1. T₂ Magnetic Resonance (T2MR) can be utilized as an end-point reading after a fixed amount of time, or a kinetic assay can be performed as described below.

Subject blood can be used with or without dilution, down to 0.5% hematocrit. Optimally the hematocrit is 2%-4%, but can be as high as 20%.

### Forward Antibody Screening

In the case of certain autoimmune conditions, antibodies can be complexed to RBCs in vivo. An assay can be performed that detects these antibodies, with an example shown in FIG. 3. Red blood cells from a donor sample are mixed with a multivalent binding agent such as anti-human globulin that can cross-link the antibodies, complement, or antibodies and complement on the surface of the RBCs and induce aggregation. T2MR can be utilized as an end-point reading after a fixed amount of time. The T₂ value can be compared to a standard curve or cut-off value to determine if aggregation occurred. This assay can also be used to confirm that laboratory prepared RBCs are not sensitized with antibodies or complement prior to use in other assays.

### Reverse Antibody Screening

A variation of the antibody screening assay or reverse blood typing assay is used to screen blood for antibodies to any of over 600 RBC antigens. An exemplary scheme of one embodiment is found in FIG. 1. This approach requires the use of specialized mixtures of donor RBCs that present, as a mixture, or singly, all known blood types. The main complicating factor in these assays is the high background of human IgG or other antibodies that are not specific for the antigens of interest. This IgG can cross-react with anti-human globulin reagent or other multivalent binding agent used to aggregate RBCs. Typically, background IgG is removed by a multi-step assay that utilizes washing.

T2MR can be incorporated into multi-step wash assays. Reagent RBCs are mixed with subject plasma or serum, incubated, washed to remove excess antibodies, and then incubated with anti-human globulin or other multivalent binding agent to aggregate any RBCs that are complexed with antibodies, complement, or both antibodies and complement. Subsequent measurement of the sample with T2MR can be used to determine the extent of RBC aggregation. The T2MR signal can be monitored as an endpoint, kinetically, or as a single or multi-peak fashion as described above for the forward blood typing assay. It is possible that weakly bound antibodies may be washed off during repeated washing steps. If this occurs but complement has been recruited to the surface of the RBCs, the binding of a multivalent binding agent such as anti-human globulin to complement followed by aggregation of the RBCs can be utilized as a method of indirect detection of antibody binding. Subsequent measurement of the sample with T2MR can be monitored as an endpoint or kinetically.

Washing RBCs, or RBC-antibody complexes, or separating the desired blood group antibodies from other immunoglobulins can be accomplished in a variety of ways. These steps can include the use of gels, gel columns, fibrin mesh, size-exclusion beads, microplate assays, centrifugation, buffer washes, and specific capture reagents.

Several different methods can be employed to isolate blood group antibodies from subject serum or plasma prior to reaction with reagent RBCs. A dried RBC reagent, or RBC ghosts, can be used to present blood group antigens to the subject samples and isolate primary antibodies of interest. Antibodies to the RBC antigens present on the ghosts will bind to the ghosts and separate from the serum or plasma. Subsequent treatment with multivalent binding antigen and additional reagent RBCs can lead to a T2MR signal indicating the presence of specific blood group antibodies. Magnetic particles can be used for separation of antibodies from serum if they are coated with blood group antigens of interest. The addition of multivalent binding agent and optionally more reagent RBCs can then aggregate the particles and RBCs yielding a positive antibody test.

Several different techniques can be used to bind excess antibodies in serum or plasma. Sepharose or other gel exclusion beads can be used to bind excess IgG after initial mixing of reagent RBCs and subject serum or plasma. Affinity chromatography can also be used to bind protein including excess antibodies.

Erythrocyes and RBC-antibody complexes can be retained and separated from excess antibodies in subject plasma or serum using several reagents and methods. Fibrin mesh can be added directly, or excess fibrinogen present in whole blood or plasma can be activated with reptilase to form a fibrin mesh that will trap any RBCs present in the sample. Synthetic gel reagents with or without anti-human globulin can also be used to trap RBCs. Flowing buffer through the mesh or synthetic gel into a solution of multivalent binding agent can remove excess human IgG while the RBCs are retained in the gel. Fluidic flow systems or centrifugation can be used to separate complexed RBCs from the fibrin mesh or gel. Magnetic particles coated with antibodies that bind RBCs via different epitopes than the blood group antigen of interest can be used to magnetically separate the RBCs and RBC-complexed blood group antibodies. Subsequent addition of multivalent binding agent can lead to blood group specific aggregation of the complexed RBCs.

Superparamagnetic particles can also be used to separate RBCs or antibodies of interest from serum or as an additional agglomerant to enhance T₂ signal. These particles can be used for separation of antibodies from serum if they are coated with blood group antigens of interest. The addition of multivalent binding agent can then aggregate the particles yielding a positive antibody test. Particles coated with antibodies that bind RBCs via different epitopes than the blood group antigen of interest can be used to magnetically separate the RBCs and associated blood group antibodies from the subject sample prior to treatment with multivalent binding agent. Particles coated with anti-human-IgG can be used to bind reagent RBCs or RBC ghosts coated in subject primary antibody.

The antibody detecting assays can be performed sequentially. First, a mixture of reagent RBCs representing all available more rare RBC antigens can be used to determine whether there are any antibodies to rarer blood types present. Subsequent tests can then be performed separately with reagent RBCs representing each rare antigen in order to discover which antibody is present in the subject serum or plasma. This can increase throughput and preserve expensive reagent RBCs.

Assays can potentially be performed without washing the RBC-antibody complexes to remove excess antibodies by adding an appropriate amount of multivalent binding agent. Multivalent binding agent is titrated to a level that binds excess IgG and then induces RBC aggregation without inducing a prozone or hook effect. To account for the differences in the amount of immunoglobulins between subjects, a titration series can be established in which increasing amounts of multivalent binding agent are added to the same sample with simultaneous monitoring of T₂ values. As the amount of multivalent binding agent increases, if the T₂ signal indicative of RBC aggregation appears, then the RBC-antibody complex is present.

If needed, methods exist for distinguishing the erythrocyte sedimentation rate (ESR) from blood group specific aggregation when performing assays. In some embodiments mixing is used to suspend RBCs during the T₂ measurement. In other embodiments a synthetic gel or fibrin mesh is used to suspend erythrocytes during T₂ measurement. Fibrinogen can also be added to the reagent RBCs to aid in normalizing T₂ signals across subjects. Additives to increase viscosity can also be used.

### Magnetic Resonance Methods

The T₂ measurement is a single measure of all spins in the ensemble, measurements lasting typically 1-10 seconds, which allows the solvent to travel hundreds of microns, a long distance relative to the microscopic non-uniformities in the liquid sample. Each solvent molecule samples a volume in the liquid sample and the T₂ signal is an average (net total signal) of all (nuclear spins) on solvent molecules in the sample; in other words, the T₂ measurement is a net measurement of the entire environment experienced by a solvent molecule, and is an average measurement of all microscopic non-uniformities in the sample.

The observed T₂ relaxation rate for the solvent molecules in the liquid sample is dominated by the magnetic particles, which for certain embodiments are RBCs, which in the presence of a magnetic field form high magnetic dipole moments. In the absence of magnetic particles, the observed T₂ relaxation rates for a liquid sample are typically long (i.e., T₂ (water)=2000 ms, T₂ (blood)=1500 ms). As particle concentration increases, the microscopic non-uniformities in the sample increase and the diffusion of solvent through these microscopic non-uniformities leads to an increase in spin decoherence and a decrease in the T₂ value. The observed T₂ value depends upon the particle concentration in a non-linear fashion, and on the relaxivity per particle parameter.

### Signal Acquisition and Processing

Standard radiofrequency pulse sequences for the determination of nuclear resonance parameters are known in the art, for example, the Carr-Purcell-Meiboom-Gill (CPMG) is traditionally used if relaxation constant T₂ is to be determined. Optimization of the radiofrequency pulse sequences, including selection of the frequency of the radiofrequency pulses in the sequence, pulse powers and pulse lengths, depends on the system under investigation and is performed using procedures known in the art.

Nuclear magnetic resonance parameters that can be obtained using the methods of the present invention include but are not limited to T₁, T₂, T₁/T₂ hybrid, T₁ᵣₕₒ, T₂ᵣₕₒ and T₂*. Typically, at least one of the one or more nuclear resonance parameters that are obtained using the methods of the present invention is spin-spin relaxation constant T₂.

As with other diagnostics and analytical instrumentation, the goal of NMR-based diagnostics is to extract information from a sample and deliver a high-confidence result to the user. As the information flows from the sample to the user it typically undergoes several transformations to tailor the information to the specific user. The methods and devices of the invention can be used to obtain diagnostic information about the hemostatic condition of a subject. This is achieved by processing the NMR relaxation signal into one or more series of component signals representative of the different populations of water molecules present, e.g., in a sample containing RBCs that are aggregating or dis-aggregating. For example, NMR relaxation data, such as T₂, can be fit to a decaying exponential curve defined by the following equation:

$$f ( t ) = {\sum }_{i = 1}^{n} {A}_{i} exp ( - t / T ( i ) ) , \qquad ( 1 )$$

where f(t) is the signal intensity as a function of time, t, Aᵢ is the amplitude coefficient for the ith component, and (T)ᵢ the decay constant (such as T₂) for the ith component. For relaxation phenomenon discussed here the detected signal is the sum of a discrete number of components (i=1, 2, 3, 4 . . . n). Such functions are called mono-, bi-, tri-, tetra- or multi-exponential, respectively. Due to the widespread need for analyzing multi-exponential processes in science and engineering, there are several established mathematical methods for rapidly obtaining estimates of Aᵢ and (T)ᵢ for each coefficient. Methods that have been successfully applied and may be applied in the processing of the raw data obtained using the methods of the invention include Laplace transforms, algebraic methods, graphical analysis, nonlinear least squares (of which there are many flavors), differentiation methods, the method of modulating functions, integration method, method of moments, rational function approximation, Padé-Laplace transform, and the maximum entropy method (see Istratov, A. A. & Vyvenko, O. F. Rev. Sci. Inst. 70:1233 (1999)). Other methods, which have been specifically demonstrated for low field NMR include singular value decomposition (Lupu, M. & Todor, D. Chemometrics and Intelligent Laboratory Systems 29:11 (1995)) and factor analysis.

There are several software programs and algorithms available that use one or more of these exponential fitting methods. One of the most widely cited sources for exponential fitting programs are those written and provided by Stephen Provencher, called “DISCRETE” and “CONTIN” (Provencher, S. W. & Vogel, R. H. Math. Biosci. 50:251 (1980); Provencher, S. W. Comp. Phys. Comm. 27:213 (1982)). Discrete is an algorithm for solving for up to nine discrete components in a multi-component exponential curve. CONTIN is an algorithm that uses an Inverse Laplace Transform to solve for samples that have a distribution of relaxation times. Commercial applications using multiexponential analyses use these or similar algorithms. In fact, Bruker minispec uses the publicly-available CONTIN algorithm for some of their analysis. For the invention described here, the relaxation times are expected to be discrete values unique to each sample and not a continuous distribution, therefore programs like CONTIN are not needed although they could be used. The code for many other exponential fitting methods are generally available (Istratov, A. A. & Vyvenko, O. F. Rev. Sci. Inst. 70:1233 (1999)) and can be used to obtain medical diagnostic information according to the methods of the present invention. Information is available regarding how the signal to noise ratio and total sampling time relates to the maximum number of terms that can be determined, the maximum resolution that can be achieved, and the range of decay constants that can be fitted. For a signal to noise ratio of ˜10⁴ the theoretical limit as to the resolution of two decay constants measured, independent of the analytical method, is a resolution δ=(Tᵢ/Tᵢ₊₁) of >1.2 (Istratov, A. A. & Vyvenko, O. F. Rev. Sci. Inst. 70:1233 (1999)). Thus it is believed that the difference between resolvable decay constants scales with their magnitudes, which is not entirely intuitive and is unlike resolution by means of optical detection. The understanding of the maximum resolution and the dependence on resolution on the signal-to-noise ratio will assist in assessing the performance of the fitting algorithm.

The methods of the invention can be used on a benchtop NMR relaxometer, benchtop time domain system, or NMR analyzer (e.g., ACT, Bruker, CEM Corporation, Exstrom Laboratories, Quantum Magnetics, GE Security division, Halliburton, HTS-111 Magnetic Solutions, MR Resources, NanoMR, NMR Petrophysics, Oxford Instruments, Process NMR Associates, Qualion NMR Analyzers, SPINLOCK Magnetic Resonance Solutions, or Stelar, Resonance Systems).

The CPMG pulse sequence used to collect data with a T2reader is designed to detect the inherent T₂ relaxation time of the sample. Typically, this is dictated by one value, but for samples containing a complex mixture of states (e.g., a sample undergoing an aggregation process or disaggregation process), a distribution of T₂ values can be observed. In this situation, the signal obtained with a CPMG sequence is a sum of exponentials. One solution for extracting relaxation information from a T2reader output is to fit a sum of exponentials in a least-squares fashion. Practically, this requires a priori information on how many functions to fit. A second solution is to use the Inverse Laplace Transform (ILT) to solve for a distribution of T₂ values that make up the exponential signal observed. Again, the results of the CPMG sequence S(t), is assumed to be the sum of exponentials:

$$S ( t ) = {\sum }_{i} {A}_{i} {e}^{- t / T {2}_{i}} , \qquad ( 2 )$$

where A, is the amplitude corresponding to the relaxation time constant T2₁. If, instead of a discrete sum of exponentials, the signal is assumed to be a distribution of T₂ values, the sum over states can be represented by:

S(t)=∫₀^{∞}A(1/T2)e^{-t/T2}d(1/T2) (3)

This has the same functional form as the ILT:

F(t)=∫₀^{∞}A(s)e^{-st}ds (4),

and can be treated as such. The ILT of an exponential function requires constraints to solve. A few methods that can be used to impose constraints are CONTIN, finite mixture modeling (FMM), and neural networks (NN). An Inverse Laplace Transform may also be used in the generation of a 3D data set. A 3D data set can be generated by collecting a time series of T₂ decay curves and applying an Inverse Laplace Transform to each decay curve to form a 3D data set. Alternatively, a 2D Inverse Laplace Transform can be applied to a pre-assembled 3D data set to generate a transformed 3D data set describing the distribution of T₂ times.

In a heterogeneous environment containing two phases, several different exchange regimes may be operative. In such an environment having two water populations (a and b), rₐ and r_{b} correspond to the relaxation rates of water in the two populations; fₐ and f_{b} correspond to the fraction of nuclei in each phase; Tₐ and T_{b} correspond to residence time in each phase; and a=(1/Tₐ)+(1/T_{b}) corresponds to the chemical exchange rate. The exchange regimes can be designated as: (1) slow exchange: if the two populations are static or exchanging slowly relative to the relaxation rates rₐ and r_{b}, the signal contains two separate components, decaying with time constants T₂₈ and T_{2b}; (2) fast exchange: if the rate for water molecules exchanging between the two environments is rapid compared to rₐ and r_{b}, the total population follows a single exponential decay with an average relaxation rate (rₐᵥ) given by the weighted sum of the relaxation rates of the separate populations; and (3) intermediate exchange: in the general case where there are two relaxation rates r₁ and r₂ with r₁ equal to rₐ in the slow exchange limit rₐ<r_{b}, Amp₁+Amp₂=1, and where r_{1,2} goes to the average relaxation rate in the fast exchange limit, equations 5, 6, 7, and 8 may be applied:

$$\begin{matrix}{r}_{1} = ( 1 / 2 ) ( {r}_{a} + {r}_{b} + a ) - ( 1 / 2 ) \sqrt{{( {r}_{b} - {r}_{a} + a )}^{2} - 4 {af}_{b} ( {r}_{b} - {r}_{a} )} & ( 5 ) \\ {r}_{2} = ( 1 / 2 ) ( {r}_{a} + {r}_{b} + a ) + ( 1 / 2 ) \sqrt{{( {r}_{b} - {r}_{a} + a )}^{2} - 4 {af}_{b} ( {r}_{b} - {r}_{a} )} & ( 6 ) \\ {Amp}_{1} = \frac{{r}_{2} - {r}_{av}}{{r}_{2} - {r}_{1}} & ( 7 ) \\ {Amp}_{2} = \frac{{r}_{av} - {r}_{1}}{{r}_{2} - {r}_{1}} & ( 8 )\end{matrix}$$

The invention also features the use of a pulsed field gradient or a fixed field gradient in the collection of relaxation rate data. The invention further features the use of the techniques of diffusion-weighted imaging (DWI) as described in Vidmar et al. (Vidmar et al., NMR Biomed. 23: 34-40 (2010)), which is herein incorporated by reference, or any methods used in porous media NMR (see, e.g., Bergman et al., Phys. Rev. E51: 3393-3400 (1995), which is herein incorporated by reference).

Other systems can be used to practice the invention, including High resolution benchtop NMR magnets and spectrometers (e.g. Magritek's ultra-compact spectromter, picospin45, NanalysisNMReady 60p cover the range of 40 MHz-60 MHz), high resolution cryogenic systems, and magnetic resonance imaging systems. With sufficient magnetic field homogeneity, NMR spectroscopy can be used to monitor the chemical shift of more than one water population in a sample during the aggregation of RBCs, particles, or RBCs and particles. Using this method, unique chemical shift signals can be associated with a tightly bound aggregate. The different chemical shifts of aggregate and non-aggregate signals arise from inherent chemical shifts of nuclei, slowing of water diffusion within a tightly bound aggregate, as well as microscopic inhomogeneities due to paramagnetic centers in heme within red blood cells. The paramagnetic effect has been shown to induce changes in chemical shift be several reports, as known in the art; such as the Evans NMR method and others (see Chu et al., Magn Reson Med, 13:239 (1990).

Alternatively, when the methods of the invention are carried out using the measurement of the T₂*, or free induction decay, rather than T₂, the relaxation properties of a specific class of, for example, water protons in the sample can be made using an off resonance radiation (i.e., radiation that is not precisely at the Larmour precession frequency). The output can be in the form of the height of a single echo obtained with a T₂ measuring pulse sequence rather than a complete echo train. In contrast, normal T₂ measurements utilize the declining height of a number of echoes to determine T₂. The T₂* approach can include the steps of shifting the frequency or strength of the applied magnetic field, and measuring the broadness of the water proton absorption peak, where broader peaks or energy absorption are correlated with higher values of T₂. The methods can be carried out using techniques for measuring water diffusion, or utilizing the slope of an echo train. In particular embodiments the measurement is made using a CPMG sequence, or a portion thereof, for example, to remove signals associated with a sample holder.

In one embodiment the average T₂ value of the water in the sample (i.e. determined from a monoexpoential fit) increases with RBC aggregation. In another embodiment, when the concentration of RBCs is high (ca. greater than half the volume of the sample), the average T₂ value decreases with aggregation of RBCs. In another embodiment, the average T₂ value increases if the majority of the water molecules are outside of the diffusion range of the RBCs. In still another embodiment, multiexponential fits are used to identify the change in T₂ for two or more water populations in the sample. In this case, the T₂ value for water around the RBCs is typically low, in the range of about 50 ms to about 300 ms.

In any of the above methods, the water in the sample can include multiple water populations, and an NMR parameter associated with a given water population can be correlated with a change in the aggregation of RBCs. For example, NMR parameter values of one water population near RBCs can be affected by the displacement of these water molecules with aggregation. Another water population in the bulk solution could be differently affected by the change in RBC aggregation state. Yet another population could exist inside the RBCs.

T2MR can be used in kinetic assays employing the methods of the invention. The T2MR signals are measured at time zero and monitored over time. The data can be collected and monitored as a “single-peak” T₂ value using a monoexponential data processing method. The average T₂ value using a monoexponential fit can go up or down relative to time zero based on the individual conditions present in the sample, including relative volume of RBCs in the sample, additives, and pre-treatment conditions. The data can also be collected and monitored using a multi-exponential method such as bi-exponential analysis, tri-exponential analysis or Inverse Laplace Transform (ILT) analysis. If the assay is done in whole blood, multiple peaks on the ILT transformed data could be observed that could represent resolution or discrimination of reacted and unreacted RBC's and serum. The T₂ signature that unfolds over time can be used to evaluate the rate of RBC aggregation. Any of the forgoing assay types or following examples can be accomplished using a kinetic format. This kinetic reading can be useful for discriminating RBC settling, which can be relatively rapid for some clinical indications, from blood-type specific aggregation caused by the antibody reagent.

NMR data is processed to create a T₂ signature curve displaying distinct signals (i.e. maxima) that represent individual water populations within a blood sample. The T₂ signature curves are created by applying a mathematical transform (e.g., a Laplace transform or Inverse Laplace Transform) to a decay curve associated with T₂ at a time point during an aggregation event.

In any of the above methods, the algorithm can include an algorithm selected from the group consisting of a multi-exponential algorithm, a bi-exponential algorithm, a tri-exponential algorithm, a decaying exponential algorithm, a Laplace transform, a goodness-of-fit algorithm, an SSE algorithm, a least squares algorithm, a non-negative least squares algorithm, or any algorithm described herein. In particular embodiments, the algorithm is an Inverse Laplace Transform.

In any of the above methods, the relaxation rate can be selected from the group consisting of T₁, T₂, T₁/T₂ hybrid, T₁ᵣₕₒ, T₂ᵣₕₒ, and T_{2*}. In one particular embodiment, the relaxation rate measurements include a T₂ measurement, and the measurement provides a decay curve.

In the aggregation assays of the invention, the number of magnetic particles (e.g., RBCs), and if present the number of agglomerant magnetic particles remain constant during the assay. The spatial distribution of the particles change when the particles cluster. Aggregation changes the average “experience” of a solvent molecule because particle localization into clusters is promoted rather than more even particle distributions. At a high degree of aggregation, many solvent molecules do not experience microscopic non-uniformities created by magnetic particles and the T₂ approaches that of solvent. As the fraction of aggregated magnetic particles increases in a liquid sample, the observed T₂ is the average of the non-uniform suspension of aggregated and single (unaggregated) magnetic particles. The assays of the invention are designed to produce a measurable change in T₂ while utilizing a reduced sample size and reagent concentration.

The red blood cells are covered in antigens that can bind with primary antibody and multivalent binding moieties in the solution to form aggregates. Aggregation depletes portions of the sample from the microscopic magnetic non-uniformities that disrupt the solvent's T₂ signal, leading to a change in T₂ relaxation.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### Example 1. Antibody Screening or Reverse Blood Typing

Briefly, the assay includes adding a small volume of reagent red blood cells (e.g. 10 μL) to a small volume (e.g. 10 μL) of patient serum or plasma. Potentiator can be added to accelerate the binding of antibodies to the surface of the reagent red blood cells. The sample can then be centrifuged and washed if desired. A multivalent binding agent such as monoclonal IgM can be added to promote aggregation. After mixing, the T₂ reading can be taken to determine whether antibodies to the blood group on the reagent blood cells is present.

The principle of this assay is that T2MR is sensitive to RBC aggregation and can measure the rate of RBC aggregation. The extent of aggregation will depend on the binding of antibodies to the antigens on the surface of the RBCs and the subsequent complexation of the multivalent binding agent that cross-links antibodies on the surface of the RBCs. Aggregation is similar to clot formation, a process that can be well-monitored by T2MR. Parameters that can be optimized include: (1) erythrocyte sedimentation rate, (2) mixing, (3) incubation, (4) sensitivity, (5) RBC level, (6) levels of multivalent binding agent, and (7) erythrocyte settling driven by other causes.

The numbers of RBCs will remain constant throughout the assay in order to avoid a dilution effect on the T₂ signal.

### Example 2. Two Channel Concept

This assay involves the comparison of two samples. One sample contains subject serum (e.g. 10 μL) and an equal volume of reagent RBCs and the other sample contains corresponding amounts of reagent RBCs combined with control serum lacking antibodies to the reagent RBCs. Comparison of the T₂ signals of the target and control samples will occur over about 10 minutes. The change in T₂ may either be the result of aggregation (presence of the settling peak, presence of a third peak, or change in the T₂ of the settling peak relative to control).

### Example 3. Direct Blood Typing for ABOD Antigens

A small volume of washed patient red blood cells (e.g. 10 μL) are obtained and mixed separately with a small volume (e.g. 10 μL) of IgM against each of the ABOD antigens. After mixing, the T₂ measurement is taken, possibly in a dynamic state with mixing. The T₂ value can be compared to a standard curve or cut-off value to determine if the RBCs did indeed aggregate.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

1. A method for identifying the presence of an analyte in a sample, said method comprising:
(a) providing red blood cells substantially free of antibodies and contacting the red blood cells with a primary antibody capable of complexing to the red blood cells when the red blood cells comprise an antigen for the primary antibody;
(b) following step (a),
(i) combining the red blood cells with an aqueous solution to form an aqueous sample, wherein the aqueous sample is substantially free of uncomplexed antibodies, and
(ii) contacting the aqueous sample with a multivalent binding agent, wherein said multivalent binding agent is operative to promote aggregation of the red blood cells when the primary antibody is complexed to the red blood cells;
(c) following step (b), placing the aqueous sample in a device, the device comprising a support defining a well configured to hold the aqueous sample, and having an RF coil disposed about the well, the RF coil configured to detect a magnetic resonance signal produced by exposing the aqueous sample to a bias magnetic field created using one or more magnets and an RF pulse sequence, and exposing the aqueous sample to a bias magnetic field and an RF pulse sequence;
(d) following step (c), measuring the magnetic resonance signal; and
(e) on the basis of the signal measured in step (d), determining whether the analyte is present, wherein the analyte is the primary antibody or a red blood cell comprising an antigen for the primary antibody.

2. The method of claim 1, wherein the red blood cells are obtained from a subject and wherein the primary antibody is a reagent primary antibody selective for an antigen found on red blood cells.

3. The method of claim 1, wherein the primary antibody is obtained from a subject, and wherein the red blood cells are reagent red blood cells that solely bear antigens for the primary antibody.

4. The method of claim 1, wherein the primary antibody is obtained from a subject, and wherein the red blood cells are reagent red blood cells bearing antigens for a mixture of primary antibodies;
wherein in part (e) if antibodies are determined to be present, the method from claim 3 is performed.

5. A method of claim 3 or 4, wherein prior to step (a) the subject primary antibodies are separated from any other plasma components.

6. A method for identifying the presence of an analyte in a sample, said method comprising:
(a) providing red blood cells substantially free of antibodies;
(b) following step (a),
(i) combining the red blood cells with an aqueous solution to form an aqueous sample, wherein the aqueous sample is substantially free of uncomplexed antibodies, and
(ii) contacting the aqueous sample with a multivalent binding agent, wherein said multivalent binding agent is operative to promote aggregation of the red blood cells when it is complexed to the red blood cells;
(c) following step (b), placing the aqueous sample in a device, the device comprising a support defining a well configured to hold the aqueous sample, and having an RF coil disposed about the well, the RF coil configured to detect a magnetic resonance signal produced by exposing the aqueous sample to a bias magnetic field created using one or more magnets and an RF pulse sequence, and exposing the aqueous sample to a bias magnetic field and an RF pulse sequence;
(d) following step (c), measuring the magnetic resonance signal; and
(e) on the basis of the signal measured in step (d), determining whether the analyte is present, wherein the analyte is a red blood cell comprising an antigen able to bind to the multivalent binding agent.

7. A method for testing the blood of a subject, said method comprising:
(a) providing a sample comprising red blood cells from said subject, said red blood cells potentially being bound with at least one of antibodies and complement factors;
(b) following step (a),
(i) combining the red blood cells with an aqueous solution to form an aqueous sample, wherein the aqueous sample is substantially free of uncomplexed antibodies and complement factors, and
(ii) contacting the aqueous sample with a multivalent binding agent, wherein said multivalent binding agent is operative to promote aggregation of the red blood cells when at least one of the primary antibody and complement factors is complexed to the red blood cells;
(c) following step (b), placing the aqueous sample in a device, the device comprising a support defining a well configured to hold the aqueous sample comprising the red blood cells and antibodies, and having an RF coil disposed about the well, the RF coil configured to detect a magnetic resonance signal produced by exposing the aqueous sample to a bias magnetic field created using one or more magnets and an RF pulse sequence, and exposing the aqueous sample to a bias magnetic field and an RF pulse sequence;
(d) following step (c), measuring the magnetic resonance signal; and
(e) on the basis of the signal measured in step (d), determining the blood disease.

8. A method of any of claims 1-7, wherein prior to step (b)(i) the red blood cells are separated from any uncomplexed antibodies.

9. A method of any of claims 1-7, wherein excess multivalent binding agent is added in step (b)(ii) to overcome the effects of any uncomplexed primary antibody.

10. A method for identifying the presence of a primary antibody in a subject serum or plasma, said method comprising:
(a) providing an aqueous sample comprising reagent red blood cells complexed with a reagent primary antibody and a selective multivalent binding agent, wherein said red blood cells form an aggregate with the primary antibody and the selective multivalent binding agent;
(b) contacting the aqueous sample with solution comprising a subject serum or plasma, wherein said subject serum or plasma contains a primary antibody that is operative to promote disaggregation of the reagent red blood cells when the subject antibody complexes the selective multivalent binding agent;
(c) placing the aqueous sample in a device, the device comprising a support defining a well configured to hold the aqueous sample, and having an RF coil disposed about the well, the RF coil configured to detect a magnetic resonance signal produced by exposing the aqueous sample to a bias magnetic field created using one or more magnets and an RF pulse sequence, and exposing the aqueous sample to a bias magnetic field and an RF pulse sequence;
(d) following step (c), measuring the magnetic resonance signal; and
(e) on the basis of the signal measured in step (d), determining whether the antibody is present.

11. The method of claim 1, 6, 7, or 10, wherein the aqueous sample comprises from 0.5-20% hematocrit.

12. The method of claim 1, 6, 7, or 10, wherein the aqueous sample comprises from 1 to 50 microliters.

13. The method of claim 1 or 10, wherein the primary antibody and the antigen for the primary antibody are characteristic of the blood type of a subject, and step (f) comprises determining the blood type of the subject.

14. The method of claim 6, wherein the antigen on the surface of the red blood cells and the multivalent binding agent selective for that antigen are characteristic of the blood type of a subject, and step (f) comprises determining the blood type of the subject.

15. The method of any claims 1-14, further comprising adding in step (b)(i), a potentiator.

16. The method of any one of claims 1-15, wherein the multivalent binding agent is monoclonal IgM.

17. The method of any one of claims 1-16, wherein the multivalent binding agent is selective for the primary antibody.

18. The method of claim 17, wherein the multivalent binding agent is an aptamer.

19. The method of claim 17, wherein the multivalent binding agent is an RNA, DNA, or XNA aptamer.

20. The method of claim 17, wherein the multivalent binding agent is a polypeptide aptamer.

21. The method of claim 17, wherein the multivalent binding agent is a peptidomimetic aptamer.

22. The method of claim 17, wherein the multivalent binding agent is a secondary antibody.

23. The method of claim 17, wherein the multivalent binding agent is a dendrimer.

24. The method of claim 17, wherein the multivalent binding agent is a Fab fragment.

25. The method of any of claim 3, 4, or 5, wherein the reagent red blood cells are single donor group ◯ red blood cells.

26. The method of claim 5, wherein the subject antibody is separated by using dried red blood cells displaying the corresponding antigen on their surfaces.

27. The method of claim 5, wherein the subject antibody is separated by using magnetic particles coated with the corresponding blood group antigen.

28. The method of any of claim 3, 4, or 10, wherein the multivalent binding agent comprises a magnetic particle.

29. The method of claim 7, wherein the red blood cells are separated by washing.

30. The method of claim 29, wherein the wash comprises centrifugation.

31. The method of claim 8, wherein the red blood cells are separated by the use of sepharose or gel size-exclusion beads.

32. The method of claim 8, the red blood cells are separated by utilizing fibrin mesh.

33. The method of claim 32, wherein the fibrin mesh is formed by adding reptilase.

34. The method of claim 8, wherein the red blood cells are separated by use of a synthetic gel.

35. The method of claim 8, wherein the red blood cells are separated via a fluidic system.

36. The method of claim 8, wherein the red blood cells are separated by utilizing magnetic particles labeled with multivalent binding agents that complex red blood cells via alternate antigens from the primary antibody.

37. The method of claim 8, wherein the red blood cells are separated by utilizing a microplate based technique.

38. The method of any one of claims 1-8, 25, and 29-37, wherein the red blood cell comprises complement and the multivalent binding agent is operative to bind to complement and induce aggregation of the red blood cells.

39. The method of any one of claims 1-15 and 38, wherein the multivalent binding agent is antihuman globulin reagent.

40. The method of any one of claims 1-9 and 11-39, wherein in step (b)(ii) the multivalent binding moiety is titrated into the red blood cells.

41. The method of any one of claims 1-40, wherein there is dynamic mixing during step (c).

42. The method of any one of claims 1-41, wherein fibrin mesh is used during step (c) to suspend the red blood cells and prevent erythrocyte sedimentation.

43. The method of any one of claims 1-42, wherein a synthetic gel is used during step (c) to suspend the red blood cells and prevent erythrocyte sedimentation.

44. The method of any one of claims 1-43, wherein an additive is added in step (c) to increase viscosity and prevent erythrocyte sedimentation.

45. The method of any one of claims 1-44, wherein the aqueous sample is centrifuged prior to step c, in order to promote aggregation.

46. The method of any one of claims 1-45, wherein the uncomplexed red blood cells are recovered.

47. The method of claim 7, wherein no multivalent binding agent is used, but red blood cells are mixed to promote rouleaux and aggregation.

48. The method of any one of claim 1, 6, or 10, wherein proteolytic enzymes are added prior to step (b)(ii).

49. The method of claims 1-48, further comprising (i) making a series of magnetic resonance relaxation rate measurements of water in said aqueous sample; (ii) transforming said measurements using an algorithm that distinguishes two or more separate water populations within said aqueous sample, wherein each separate water population is characterized by one or more magnetic resonance parameters having one or more values; and (iii) on the basis of the transformed measurements of step (ii), determining the presence and identity of antigens on the surface of red blood cells in the blood sample and additionally or alternatively determining the presence of antibodies in the aqueous sample.
